(19) United States
(12) Patent Application Publication
(10) Pub. No.: US 2016/0030245 A1
(43) Pub. Date: Feb. 4, 2016

(54) DEVICES AND METHODS FOR SUPPRESSING TINNITUS

(71) Applicant: SOUNDCURE, INC., San Jose, CA (US)

(72) Inventors: William A. Perry, Sunnyvale, CA (US); Jeffrey A. Carroll, Corona, CA (US)

(21) Appl. No.: 14/777,421

(22) PCT Filed: Mar. 15, 2013
(86) PCT No.: PCT/US13/31904

§ 371 Date: Sep. 15, 2015

(51) Int. Cl. A61F 11/00; A61B 5/12; H04R 25/00; A61N 1/36

(52) U.S. Cl. CPC A61F 11/00; A61N 1/36032; A61B 5/128; A61N 1/361; A61N 1/36132; H04R 25/75

(57) ABSTRACT

Methods and devices for treating tinnitus in a human subject wherein the subject is caused to perceive a tinnitus suppressing sound which fully or partially suppresses the subject's tinnitus. The tinnitus suppressing sound can be set to a pitch offset from a matched pitch for the human subject. The offset can be one or more half octaves or octaves lower or higher than the matched pitch. The offset can also be set as a function of the human subject's hearing loss and as a function of limitations in the sound producing device so that the optimal sound for the human subject when wearing the sound producing device is the sound that is used in that situation. The produced sounds may also be modulated with a sinusoid for improved suppression. Other methods and devices are also disclosed.

### FIELD OF THE INVENTION

The present invention relates generally to devices and methods for the treatment of tinnitus.

### BACKGROUND OF THE INVENTION

Tinnitus is the perception of sound in the absence of corresponding external sounds. Tinnitus may be caused by injury, infection, the repeated bombast of loud sound or anything that can damage or alter a person's hearing including the normal aging process, and can appear in one or both ears. Although known for its high-pitched ringing, tinnitus is an internal noise that varies in its pitch and frequency. The sound perceived may range from a quiet background noise to a signal that is perceived as very loud.

Tinnitus occurs in the setting of sensorineural hearing loss in the majority of patients, thus the postulate that tinnitus is triggered peripherally in the cochlea. Noise damage causes molecular changes to structural proteins in stereocilia and the cuticular plate. Cytoplasmic calcium levels increase dramatically in response to sound, potentially disrupting normal hair cell function. Progressive insult results in complete destruction of hair cells in certain regions of the basilar membrane. Aberrant auditory signals occur around areas of hair cell loss, an edge effect that results in the perception of sound. Furthermore, auditory nerve fibers are spontaneously active during quiet, resulting in neurotransmitter release. Loss of the spontaneous activity can lead to abnormal central auditory activity, which could be perceived as sound. Lack of sound input and edge effects as a cause of tinnitus could explain the reduction of tinnitus commonly seen following cochlear implantation.

As shown in prior art PCT Application No. WO2009/076191 A1 entitled, “Devices and Methods for Suppression of Tinnitus” (“the '191 Application”), tinnitus can be separated into three categories depending on the severity and whether hearing loss is present. Category 0 is characterized by the tinnitus having a low impact on the person's life. Categories 1 and 2 are used to describe tinnitus with a high impact on life with Category 2 indicating the presence of subjective hearing loss that accompanies the tinnitus. The '191 Application followed this convention, but a second parameter based on loudness was also defined. The loudness parameter was determined by each subject as Low, Moderate or High. On a 10-point scale with 1 being the lowest sound (threshold) and 10 being the upper limit of loudness, Low is 0 to 3, Moderate is 4 to 6, and High is 7 to 10.

FIG. 1 is a graphic representation of this tinnitus severity classification and the typical tinnitus patient population. As explained in the '191 Application, patients within category 0 are least likely to seek tinnitus treatment. Category 1 and 2 patients with low levels of loudness are likely to be helped by TRT. It has been shown that TRT helps in relaxation for a majority of patients (72.5%) but benefits a much smaller percentage with respect to their ability to work (25.5%) and sleep (47%). At present, category 1 and 2 patients with high levels of loudness are often left without effective treatment.

Several manufacturers provide an earpiece that can generate a masking sound. Masking methods work well for people who suffer mild forms of tinnitus, but do not work for people who suffer from loud tinnitus because, to mask the tinnitus, the external sound has to be louder than the tinnitus. Some treatments exist, such as drugs, surgery, and psychotherapy, but none are consistently effective and may have significant side effects.

The '191 Application presents techniques for suppressing tinnitus. However, given the limitations in commercial sound producing devices alone or in combination with the hearing loss in many subject patients, patients continue to suffer from tinnitus.

There remains a need in the art for the development of new devices and methods for the treatment of tinnitus.

### SUMMARY OF THE INVENTION

The present invention provides methods and devices for suppressing tinnitus.

In accordance with one aspect, there is provided a method of generating sound for treating a tinnitus condition in a human subject. The method includes adjusting a frequency of a presented sound as a function of feedback from the human subject to generate an adjusted sound comprising at least a matched pitch corresponding to the tinnitus condition. The method also includes generating a sound at a preselected pitch offset from the matched pitch. The generated sound provides a tinnitus-suppressing sound for the human subject. The preselected pitch may be offset one or more octaves or half octaves lower or higher than the matched pitch. The generated sound may be modulated with a sinusoid to generate a modulated sound. The portable sound generating device may be a hearing aid, an acoustic ear device, an earpiece, a headset, a speaker, a cochlear implant or an implanted electrode.

In accordance with another aspect, there is provided a method of generating sound for treating a tinnitus condition in a human subject. The method includes adjusting a frequency of a presented sound as a function of feedback from the human subject to generate an adjusted sound comprising at least a matched pitch corresponding to the tinnitus condition. The method also includes positioning a portable sound generating device on the human subject and generating a modified sound with the portable sound generating device at a preselected pitch offset from the matched pitch as a function of feedback from the human subject. The modified sound provides a tinnitus-suppressing sound for the human subject. The preselected pitch may be offset from the matched pitch to the nearest audible pitch generated by the portable sound generating device based on feedback from the human subject. The nearest audible pitch may be the nearest audible pitch bearing a similarity to the matched pitch based on feedback from the human subject. The preselected pitch may be offset from the matched pitch to the nearest audible pitch generated by the portable sound generating device as a function of a hearing limitation in the human subject and as a function of a sound generation limitation in the portable sound generating device.

In accordance with yet another aspect, there is provided a device for generating sound for treating tinnitus in a human subject. The device includes a portable sound generating device adapted to be positioned on the human subject and a processor that executes instructions for adjusting a frequency of a sound presented to the human subject as a function of feedback received from the human subject to generate an adjusted sound comprising a matched pitch. The portable sound generating device generates a sound at a preselected pitch offset from the matched pitch. The generated sound provides a tinnitus-suppressing sound for the human subject. The preselected pitch may be offset one or more octaves or half octaves lower or higher than the matched pitch. The device may be a hearing aid, an acoustic ear device, an earpiece, a headset, a speaker, a cochlear implant or an implanted electrode for producing a sound as a function of the generated sound or of the modulated sound.

In accordance with yet still another aspect, there is provided a device for generating sound for treating tinnitus in a human subject. The device includes a portable sound generating device adapted to be positioned on the human subject and a processor that executes instructions for adjusting a frequency of a sound presented to the human subject as a function of feedback received from the human subject to generate an adjusted sound comprising a matched pitch. The portable sound generating device generates a modified sound at a preselected pitch offset from the matched pitch and the preselected pitch is preselected as a function of sound generated by the portable sound generating device and feedback from the human subject. The modified sound provides a tinnitus-suppressing sound for the human subject. The preselected pitch may be offset from the matched pitch to the nearest audible pitch generated by the portable sound generating device based on feedback from the human subject. The nearest audible pitch may be the nearest audible pitch bearing a similarity to the matched pitch based on feedback from the human subject. The preselected pitch may be offset from the matched pitch to the nearest audible pitch generated by the portable sound generating device as a function of a hearing limitation in the human subject and as a function of a sound generation limitation in the portable sound generating device.

In accordance with yet still another aspect, there is provided a device for generating sound for treating tinnitus in a human subject having a known matched pitch associated with the tinnitus. The device includes a portable sound generating device adapted to be positioned on the human subject and electrical connections within the portable sound generating device for generating a varying sound at a pitch offset from the matched pitch. The device also includes an input for setting a pitch as a function of the varying sound generated by the portable sound generating device and feedback from the human subject. The portable sound generating device produces a modified sound as a function of the set pitch. The modified sound provides a tinnitus-suppressing sound for the human subject. The electrical connections may include an electrical lead adapted for receiving a signal corresponding to the varying sound. The electrical connections may include a circuit for producing a signal corresponding to the varying sound. The portable sound generating device may modulate the generated sound with a sinusoid to generate a modulated sound. The portable sound generating device may include a hearing aid, an acoustic ear device, an earpiece, a headset, a speaker, a cochlear implant or an implanted electrode for producing the modified sound or the modulated sound. The device may generate the sound at the preselected pitch offset one or more octaves or half octaves lower or higher than the matched pitch.

Other objects and features will be in part apparent and in part pointed out hereinafter.

### BRIEF DESCRIPTION OF THE DRAWINGS

FIG. 1 is a graphic representation of tinnitus severity classifications and a typical tinnitus patient population.

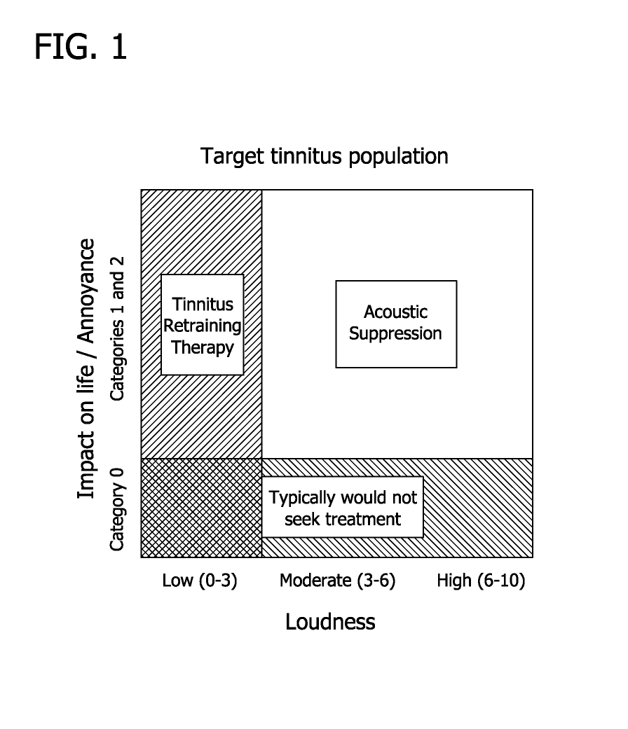

FIG. 2 is a graphic illustration showing differences between tinnitus masking and tinnitus suppression.

FIG. 3 is a diagram disclosing both devices and methods for suppressing tinnitus.

### DETAILED DESCRIPTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings are not intended to limit the scope of the invention.

When tinnitus is masked, as in the prior art, the tinnitus sound is covered with a masking sound, such as a white noise or band passed noise, that is equal to or greater in volume than the tinnitus sound. Thus, when tinnitus is masked, the subject's sound environment is the same or louder than listening to their tinnitus alone since the masking sound must be at least equal in volume to the tinnitus sound in order to completely cover the tinnitus sound. If the masker is presented quieter than the tinnitus, a case known as partial masking occurs. As the name indicates, this is when the tinnitus is partially covered so it appears lower in volume but the presence of the partial masker makes the total sound environment approximately the same. The present invention suppresses tinnitus by providing to the subject a tinnitus suppression sound (which may be an external acoustic sound or electrical cochlear or neural stimulation that corresponds to the desired tinnitus suppression sound). The tinnitus suppression sound may be lower in volume than the tinnitus and will substantially or completely eliminate the subject's perception of the tinnitus. As a result, the subject hears only the lower volume suppression sound. Since this suppression sound may be softer than the tinnitus, the total sound environment may be decreased by use of the present invention.

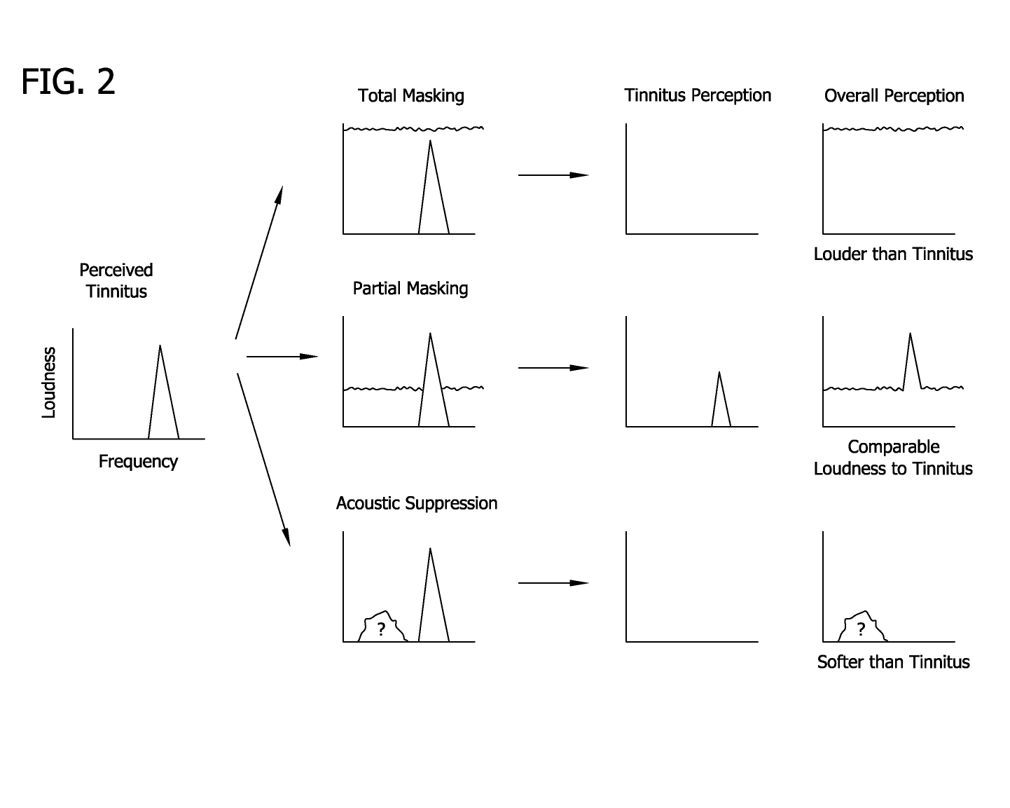

FIG. 2 shows graphically the differences between tinnitus masking and tinnitus suppression. With total masking, the tinnitus will not be audible, but the masker will be louder than the tinnitus. For partial masking, the masker is softer than the tinnitus, the perception of the tinnitus is reduced, but the overall level of sound (masker plus tinnitus) is similar to the tinnitus alone. For suppression, a sound is presented that is softer than the tinnitus but eliminates or diminishes the perception of the tinnitus. The overall level will be less than the tinnitus alone.

The present invention includes a sound delivery device and method to suppress tinnitus. The device is any device that is useable to deliver a tinnitus suppressing sound to the human subject. Such sound may be delivered acoustically via an audio device (e.g., stereo or mono sound emitting device with speaker(s) (e.g., speakers, earpiece(s), headphone(s), etc.) or electrically via an electrode or electrode array, such as a needle electrode, ear implant, cochlear implant, etc. The sound can be static or dynamic, including pure tones, click trains, amplitude-modulated and frequency-modulated sounds as well as speech and music.

After the tinnitus suppressing sound has been selected, it is delivered to the subject in a series of treatments or continuously to effect suppression of the subject's tinnitus. The tinnitus suppressing sound may be delivered in the form of acoustic sound (e.g., via speakers, earphones, headset, ear buds, ear canal inserted speakers, hearing aids, etc.) or as electrical stimulation to the cochlea, auditory nerve or appropriate area of the brain. Non-limiting examples of cochlear implants and implantable electrodes that may be used to deliver tinnitus suppressing treatments of the present invention are described in the prior art U.S. Patent Application Publication 2007/0203536 (Hochmair et al.).

Also, optionally, the loudness of the tinnitus suppressing sound may be adjusted to be softer than the tinnitus, thereby allowing the subject to avoid being subjected to an unnecessarily high perceived sound environment. The tinnitus suppressing sound may be delivered as acoustic sound or as electrical stimulus, such as electrical stimuli delivered via a cochlear implant.

A rebound increase in tinnitus can occur in some subjects after the offset of an acoustic or electric tinnitus suppressing sound. In some cases, the rebound can persist for hours when a suppressor is used upwards of 8 hours. One method to alleviate the rebound is to produce an offset ramp of approximately one minute in the suppressor. The offset ramp gradually decreases the amplitude of the tinnitus suppressing sound over a 1 minute or longer period. These event-related evoked potentials can be used to derive a tinnitus “signature” that can then be used to objectively identify the presence and absence of tinnitus.

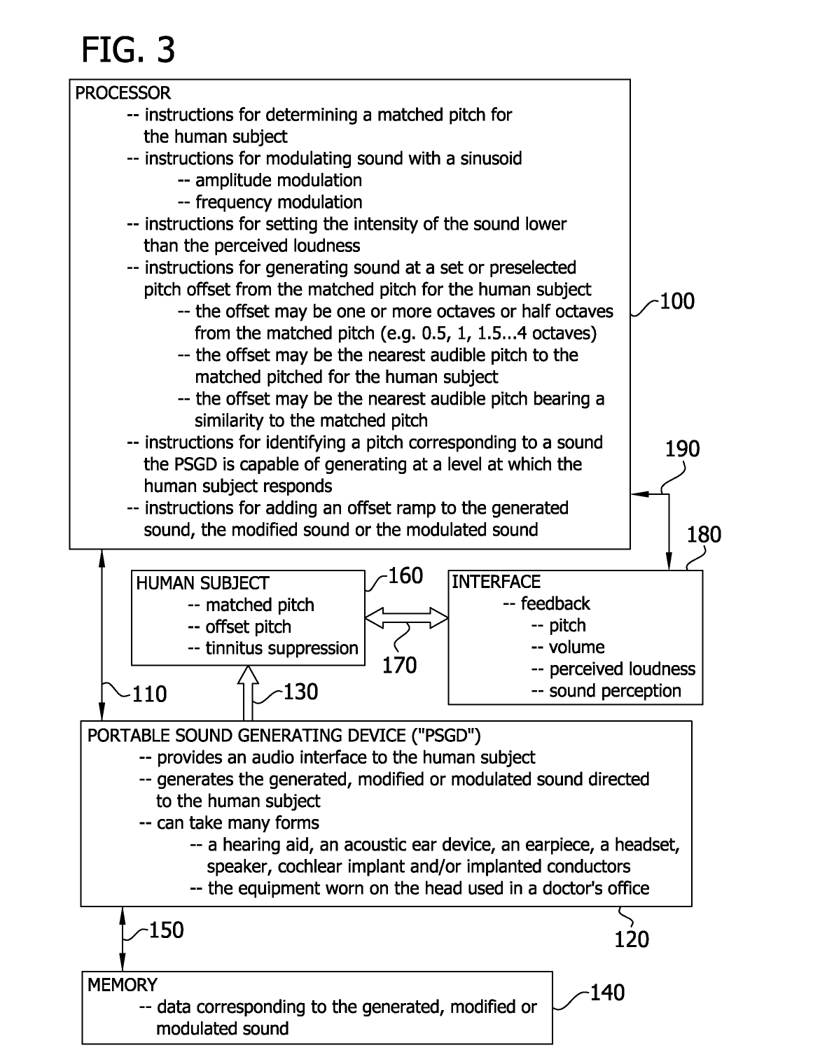

FIG. 3 is a diagram that discloses devices and methods for suppressing tinnitus. FIG. 3 broadly discloses a processor 100, a portable sound generating device (“PSGD”) 120, a memory 140, a human subject 160 and an interface 180. The processor 100 contains tangible computer readable medium storing a rules engine of computer executable instructions executable by the processor 100. These executable instructions are more fully set forth below. The PSGD 120 provides an audio interface to the human subject 160 and is typically worn by the human subject 160 during adjustment and fitting as well as during use. The PSGD 120 is used to generate all sounds heard by the human subject 160 including sounds with a varying pitch during fitting and adjustment as well as the generated sounds, modified sounds, and modulated sounds disclosed herein. The sounds are controlled by the processor 100 which supplies appropriate data and drive signals over a line 110 for use by PSGD 120 in generating the corresponding sounds.

As a matter of form factor, the processor 100 can be included as a part of the PSGD 120 and the memory 140. Such an integrated device can be manufactured as a hearing aid with all of the circuitry contained within the housing of the hearing aid. Similar commonly found electronic devices for producing sounds through ear buds, earpieces, a headset, a cochlear implant, an implanted electrode, a speaker or any other acoustic ear device can also be used and contain the processor 100, PSGD 120 and memory 140 in the same device. Likewise, the processor 100 can be located as a discrete component in a doctor's office (or similar facility) with electrical connections (e.g. line 110) between the processor 100 and the PSGD 120. In this form and as indicated by the arrow 130, the PSGD 120 (such as a conventional hearing aid, ear bud, earpiece, headset, speaker, any other acoustic ear device, a cochlear implant or an implanted electrode) can be worn by the human subject 160 while the matched pitch for the tinnitus condition is determined through use of the processor 100. The set and preselected pitch offsets described herein can also be determined while the user is wearing the PSGD 120. Likewise, the generated sounds, modified sounds and modulated sounds described herein can also be tested and adjusted while the PSGD 120 is being worn by the human subject. The fact that the human subject 160 is wearing the PSGD during the fitting and adjustment of the acoustic performance of the PSDG 120 helps to compensate for distortion and/or acoustic performance variations caused by the PSGD or caused by the fit between the human subject 160 and the PSGD 120.

During the adjustment and fitting process, the human subject 160 hears sounds through the PSGD 120, views the interface 180, and provides feedback through the interface 180 as indicated by the arrow 170. Such feedback includes the human subject's perception of the applied sound from the PSGD 120 concerning the sound's pitch, volume, perceived loudness, or any other sound characteristic where feedback from the human subject concerning the perception of the applied sound is needed. Data concerning the human subject 160's feedback is input at the interface 180 and provided to the processor 100 over a line 190.

Using the described circuitry according to the methods disclosed herein, a matched pitch for the human subject's tinnitus condition, an appropriate offset pitch and a corresponding tinnitus suppression sound can be determined Data corresponding to the generated sounds, modified sounds, and modulated sounds disclosed herein can be stored in the memory 140 via data line 150. As a matter of form factor, the memory 140 can be included with the package comprising the PSGD 120 or it can be included with the package comprising the processor 100. When the memory 140 is included with the processor 100, then the data line 150 directly connects (not shown) the processor 100 to the memory 140.

The computer instructions executable by the processor 100 include instructions for carrying out the protocols for determining the matched pitch for the human subject's tinnitus condition. Techniques for determining a matched pitch for the tinnitus condition in a human subject 160 are known in the art. As one example, such a tinnitus matching tone may be obtained by presenting an external tone to the subject through PSGD 120 who is instructed to adjust the tone's amplitude and frequency through interface 180 to match the perceived tinnitus' loudness and pitch. The tinnitus matching tone is the most accurately obtained by a double-bracketing procedure in which the amplitude of the external tone is first presented to be much softer than the perceived tinnitus loudness and then to be much louder; the range of the softer and louder amplitudes is reduced until the external tone is just noticeably softer or louder than the perceived tinnitus loudness. The average of the just noticeable softer and louder amplitudes for the external tone is the matched tinnitus loudness. Once the matched tinnitus loudness is obtained, the frequency of the external tone is varied to be much lower and higher than the perceived tinnitus pitch. Similarly, the range of the frequencies is reduced until the external tone is just noticeably lower or higher than the perceived tinnitus pitch. The average of the noticeable lower and higher frequencies for the external tone is the matched tinnitus pitch.

The computer instructions executable by the processor 100 also include instructions for generating sound at a set or preselected pitch offset from the matched pitch for the tinnitus condition in the human subject 160. For example, these computer instructions include instructions that can be selected for generating a sound based on a pitch offset lower or higher than the matched pitch by one or more octaves or half octaves, e.g., 0.5, 1, 1.5, 2, 2.5, 3, 3.5 or 4 octaves higher or lower than the matched pitch. Shifting to a lower pitch is more common because most people with hearing loss have more loss at higher frequencies (although not always as some do have other types of losses suggesting an upward shift in pitch). Shifting the pitch between octaves is relatively straightforward. Multiplying by an integer raises the pitch a corresponding number of octaves and dividing successively by the integer “two” lowers the pitch a corresponding number of octaves. For example, with a matched pitch of 8,000 Hz, a one octave upward shift in pitch would be 16,000 Hz and a one, two, three and four octave downward shift in pitch would be 4,000 Hz, 2,000 Hz, 1,000 Hz and 500 Hz, respectively. When a human subject 160 has suffered a hearing loss near the matched pitch and/or when the particular PSGD being worn by the human subject lacks sufficient frequency response to provide a suitable sound level at the matched pitch and/or when the matched pitch is simply uncomfortable or bothersome for the human subject 160, then the preferred shift is to shift the pitch to the closest octave available. Upon identification of the appropriate octave or half octave shift via feedback from human subject 160 through interface 180, data corresponding to such identified shift in pitch and generated sound is then stored in memory 140 for subsequent use in generating such tinnitus suppressing sound for human subject 160.

The rationale for shifting by octaves is twofold. First, many natural sounds, such as a ringing bell, have many frequency components, not just one, and these components are often related by octave intervals. As many sounds have this relationship, the human brain has developed processing strategies in which sounds with octave spacing interact with some of the same neurons. So by shifting through an octave, there is similar neuron activity and response to the sound stimulus. Second, it is quite common for human subjects who are undergoing a tinnitus pitch match to experience “octave confusion,” a phenomenon in which two tones space an octave apart are both judged equally similar to their tinnitus, but now values between these two octave endpoints are more similar.

The computer instructions executable by the processor 100 also include instructions for generating sound at a set or preselected pitch offset from the matched pitch for the tinnitus condition in the human subject 160 to the nearest audible pitch to the matched pit or, more preferably, to the nearest audible pitch bearing a similarity to the matched pitch. Starting with the matched pitch, the computer instructions provide that the pitch and correspondingly generated sound are varied until such nearest audible pitch is identified through feedback from the human subject 160 through interface 180. Data corresponding to such identified pitch and generated sound is then stored in memory 140 for subsequent use in generating such tinnitus suppressing sound for human subject 160.

The computer instructions executable by the processor 100 also include instructions for carrying out the protocols for determining the loudness of the tinnitus condition perceived by a human subject 160 and then setting the intensity of the tinnitus suppression sound below that perceived loudness level.

The computer instructions executable by the processor 100 also include instructions for comparing the frequency response of a hearing impaired human subject 160 to the frequency response and corresponding sound level available from the particular PSGD 120 worn by the human subject 160 to identify a pitch corresponding to a sound the PSGD 120 is capable of generating at a level at which the human subject 160 responds to the tinnitus suppression treatment. The computer instructions store data corresponding to that identified pitch and/or the corresponding generated sound for use in driving the PSGD to generate said sound for the human subject 160.

The computer instructions executable by the processor 100 also include instructions for selectively adding an offset ramp to the generated sounds, modified sounds and modulated sounds disclosed herein. Again, this helps to avoid problems with rebound at the end of a treatment.

In use, the devices disclosed herein provide for the practice of many different methods of treating a tinnitus condition in human subject 160. For example, one such method comprises generating a sound with the PSGD 120 positioned on the human subject 160. The generated sound is a tinnitus-suppressing sound for the human subject 160 and has a preselected pitch offset from the matched pitch. If desired, the generated sound can be amplitude or frequency modulated with a sinusoid to generate an amplitude or frequency modulated sound for treating the tinnitus condition. In further practice of the method, the preselected pitch can be offset to a higher or lower frequency by half or full octaves, as described above. Alternatively, the preselected pitch can be offset from the matched pitch to the nearest audible pitch, preferable the nearest audible pitch bearing a similarity to the matched pitch, generated by the PSGD 120 based on feedback from the human subject 160 through interface 180. As another alternative, the preselected pitch can be offset from the matched pitch to the nearest audible pitch generated by the PSGD 120 as a function of a hearing limitation in the human subject 160 and as a function of a sound generation limitation in the PSGD 120. An offset ramp can preferably be added to the sound provided to the human subject 160 at the end of the treatment session. Methods can also include receiving additional feedback from the human subject 160 through interface 180 after applying a generated sound, modified sound or modulated sound to the human subject 160 and then, as a function of such additional feedback, adjusting an intensity of such applied sound within a range from zero to less than the loudness perceived by human subject 160.

In practice, different devices can be constructed by assembling the different hardware components shown in FIG. 3 in whatever configuration is desired. For example, a device for treating tinnitus in a human subject 160 with a generated, modified or modulated sound can include PSGD 160 positioned on human subject 160. Processor 100 can then execute instructions for causing PSGD 120 to generate sound at a set or preselected pitch offset from the human subject's matched pitch to suppress the tinnitus condition. Processor 100 can execute instructions for causing PSGD 120 to generate sound at a set or preselected pitch offset to a higher or lower frequency by half or full octaves, as described above. Alternatively, the set or preselected pitch can be offset from the matched pitch to the nearest audible pitch, preferably the nearest audible pitch bearing a similarity to the matched pitch, generated by the PSGD 120 based on feedback from the human subject 160 through interface 180. As another alternative, the set or preselected pitch can be offset from the matched pitch to the nearest audible pitch generated by the PSGD 120 as a function of a hearing limitation in the human subject 160 and as a function of a sound generation limitation in the PSGD 120. Processor 100 can also execute instructions for modulating the amplitude or frequency of the sound with a sinusoid to generate an amplitude or frequency modulated sound to suppress the tinnitus condition in the human subject 160. After the human subject 160 has received sound from the PSGD 120, the processor 100 can execute further instructions for receiving additional feedback from the human subject 160 through the interface 180 and for adjusting the intensity of such sound as a function of the additional feedback so that the intensity falls within a range from zero to less than the perceived loudness. An offset ramp can preferably be added to the sound provided to the human subject 160 at the end of the treatment session.

When the processor 100 is not included in the form factor containing the PSGD 120, then the settings and adjustments to vary the sound output by the PSGD 120 from the matched pitch are made by processor 100 through an electrical connection 110 between PSGD 120 and processor 100. The PSGD 120 includes an input for setting the pitch as a function of the varied sound to provide a tinnitus suppressing sound offset from the matched pitch for the human subject 160. After the settings and adjustments are made, the electrical connection 110 is separated (as by unplugging a line) and the circuitry needed for producing a signal corresponding to the varying tinnitus suppressing sound remains with the form factor for the PSGD 120 and makes electrical connection through the input to connector 110. The set pitch can be offset to a higher or lower frequency by half or full octaves, as described above. Alternatively, the set pitch can be offset from the matched pitch to the nearest audible pitch, preferably the nearest audible pitch bearing a similarity to the matched pitch, generated by the PSGD 120 based on feedback from the human subject 160 through interface 180. As another alternative, the set pitch can be offset from the matched pitch to the nearest audible pitch generated by the PSGD 120 as a function of a hearing limitation in the human subject 160 and as a function of a sound generation limitation in the PSGD 120. The amplitude or frequency of the tinnitus suppressing sound can be modulated with a sinusoid to generate an amplitude or frequency modulated sound to suppress the tinnitus condition in the human subject 160. An offset ramp can preferably be added to the sound provided to the human subject 160 at the end of the treatment session.

It is to be appreciated that the invention has been described hereinabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

1. A method of generating sound for treating a tinnitus condition in a human subject, comprising the steps of:
adjusting a frequency of a presented sound as a function of feedback from the human subject to generate an adjusted sound, the presented sound before adjustment comprising at least a matched pitch corresponding to the tinnitus condition; and
generating the adjusted sound at a preselected pitch offset from the matched pitch;
wherein the generated sound is a tinnitus-suppressing sound for the human subject.

2.-3. (canceled)

4. The method of claim 1 wherein the generating step further comprises generating the adjusted sound with a portable sound generating device adapted to be positioned on the human subject.

5. The method of claim 4 wherein the portable sound generating device comprises a hearing aid, an acoustic ear device, an earpiece, a headset, a speaker, a cochlear implant or an implanted electrode.

6.-7. (canceled)

8. The method of claim 1 wherein the preselected pitch is offset one or two octaves lower than the matched pitch.

9. The method of claim 1 wherein the preselected pitch is offset three or four octaves lower than the matched pitch.

10. (canceled)

11. The method of claim 1 wherein the preselected pitch is offset one octave higher than the matched pitch.

12. The method of claim 1 wherein the preselected pitch is offset one half octave higher than the matched pitch.

13. (canceled)

14. The method of claim 1 wherein the adjusting step comprises adjusting the frequency of the presented sound including reducing a range of frequencies until the matched pitch is reached, wherein said range of frequencies varies from lesser than to greater than a perceived pitch.

15.-17. (canceled)

18. The method of claim 1 further comprising applying the adjusted sound to the human subject, thereby suppressing the tinnitus of the human subject.

19. (canceled)

20. The method of claim 1 further comprising adding an offset ramp to the adjusted sound.

21. The method of claim 1 further comprising storing data corresponding to the adjusted sound in a portable audio device.

22. The method of claim 1 wherein the adjusted sound has an amplitude and wherein the generating step further comprises generating the adjusted sound to generate an amplitude modulated sound at a reselected itch offset from the matched itch or the adjusted sound has a frequency and wherein the generating step further comprises generating the adjusted sound to generate a frequency modulated sound at a preselected pitch offset from the matched pitch.

23.-76. (canceled)

77. A device for generating sound for treating tinnitus in a human subject having a known matched pitch associated with the tinnitus, the device comprising:
a portable sound generating device adapted to be positioned on the human subject; and
electrical connections within the portable sound generating device for generating a varying sound at a pitch offset from the matched pitch; and
an input for setting a pitch to provide a set itch as a function of the varying sound generated by the portable sound generating device and feedback from the human subject;
wherein the portable sound generating device produces a modified sound as a function of the set pitch; and
wherein the modified sound is a tinnitus-suppressing sound for the human subject.

78.-80. (canceled)

81. The device of claim 77 wherein the portable sound generating device further comprises a hearing aid, an acoustic ear device, an earpiece, a headset, a speaker, a cochlear implant or an implanted electrode for producing the modified sound or the modulated sound.

82. (canceled)

83. The device of claim 77 wherein the set pitch is offset one half octave lower than the matched pitch.

84. The device of claim 77 wherein the set pitch is offset one or two octaves lower than the matched pitch.

85. The device of claim 77 wherein the set pitch is offset three or four octaves lower than the matched pitch.

86. (canceled)

87. The device of claim 77 wherein the set pitch is offset one octave higher than the matched pitch.

88. The device of claim 77 wherein the set pitch is offset one half octave higher than the matched pitch.

89. The device of claim 77 wherein the set pitch is offset from the matched pitch to the nearest audible pitch generated by the portable sound generating device based on feedback from the human subject.

90. (canceled)

91. The device of claim 77 wherein the set pitch is offset from the matched pitch to the nearest audible pitch generated by the portable sound generating device as a function of a hearing limitation in the human subject and as a function of a sound generation limitation in the portable sound generating device.

92. The device of claim 77 wherein the modified sound has an amplitude and wherein the portable sound generating device further comprises a modulator to modulate the amplitude of the modified sound to generate an amplitude modulated sound, wherein the portable sound generating device generates the amplitude modulated sound as a tinnitus-suppressing sound for the human subject; or the modified sound has a frequency and wherein the portable sound generating device further comprises a modulator to modulate the frequency of the modified sound to generate a frequency modulated sound, wherein the portable sound generating device generates the frequency modulated sound as a tinnitus-suppressing sound for the human subject.

93. (canceled)

94. The device of claim 77 wherein the portable sound producing device adds an offset ramp to the modified sound.
